(19) United States
(12) Patent Application Publication
(10) Pub. No.: US 2002/0177815 A1
(43) Pub. Date: Nov. 28, 2002

(54) Angiographic guide wire with tether

(76) Inventor: Cicero H. Malilay, Los Angeles, CA (US)

Correspondence Address: Linval B. Castle, Suite 260, 22693 Hesperian Blvd., Hayward, CA, 94541-7046, US

(21) Appl. No.: 09/864,480

(22) Filed: May 25, 2001

(51) Int. Cl. A61M 5/178

(52) U.S. Cl. 604/170.01

(57) ABSTRACT

An angiographic guide wire with a tether coupled to one end so that a guide wire unwittingly lost in an artery may be easily and quickly withdrawn.

### BACKGROUND OF THE INVENTION

This invention relates to surgical equipment and in particular to a guide wire for guiding a softer catheter into a vein or artery.

A catheter is usually difficult to insert into a blood vessel because it is soft and easily bent so that it loops on itself. Therefore a guide wire is used to guide the soft catheter and to insure a smooth insertion. These angiographic guide wires are actually fine stainless steel wires tightly wound as a helix on a very small core wire for a controlled amount of flexibility. The smallest guide wire for use in small blood vessels has an overall diameter in the order of ½ mm. and a length of approximately 15 cm. They are available from surgical suppliers throughout most of the world.

A complication that may occur while attempting to insert a catheter into a blood vessel such as an arterial line for blood pressure monitoring, is for the operator to unwittingly push the entire guide wire into the blood vessel. While uncommon, this accident occasionally occurs when the operators are tired and overworked residents. The accident won't help the operator's sleep; he will lay awake with visions of a guide wire lost in the patient's arteries. Retrieval of the guide wire and catheter from the patient requires the expertise of a surgeon or a cardiologist and involvement of the radiology department plus the expense of ancillary services.

To prevent this accident from occurring, this invention is for a guide wire which has been redesigned to include a short length of tether wire extending from one end so that an operator can see that he is already at the end of the guide wire or, if totally pushed into a blood vessel, the operator can easily redraw the guide wire by pulling on the tether.

### BRIEF DESCRIPTION OF THE DRAWINGS

In the drawings that illustrate the preferred embodiment of the invention:

FIG. 1 is an enlarged view of the angiographic guide wire with tether;

FIG. 2 is an enlarged view of the tethered end of the guide wire of FIG. 1; and

FIG. 3 is a sectional view of the tethered end of the guide wire of FIG. 2.

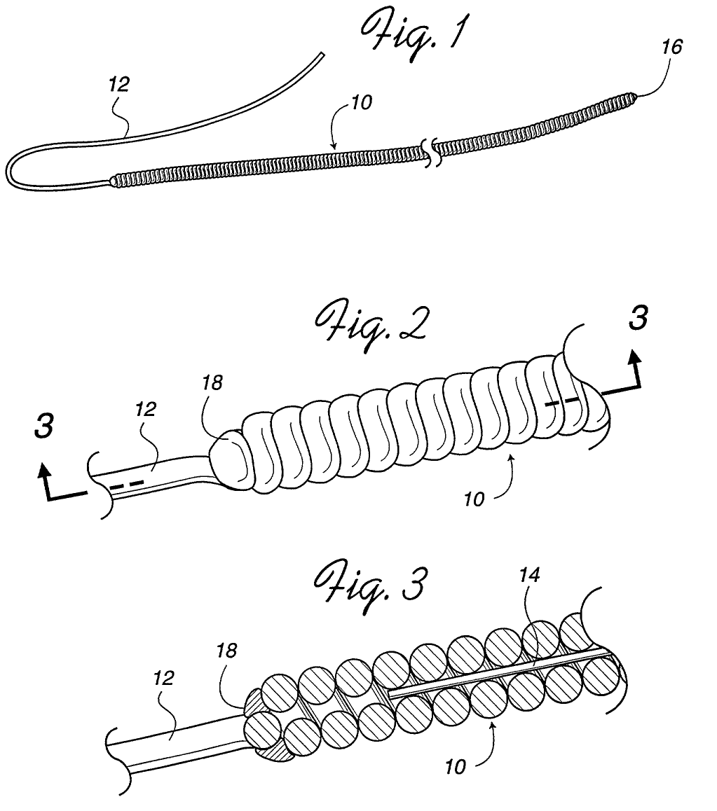

### DETAILED DESCRIPTION OF THE PREFERRED EMBODIMENT

The drawings illustrate an angiographic guide wire 10 with a tether wire 12 added at one end. The angiographic guide wire 10 without tether is a well known commodity that is readily available from surgical suppliers throughout the world and is used to help insert catheters and other small instruments into veins and arteries. Thus, the guide wire 10 must necessarily have a very small diameter and must be stiff, yet sufficiently flexible so that it can follow the natural curves of the veins. They achieve these properties by spiral winding a very fine stainless steel wire around a vary fine stainless steel straight core 14, the total overall diameter of which is only about ½ mm.

One end 16 of the angiographic guide wire 10 is blunt for contact with the catheter and the opposite end 18 is controlled by the operator for inserting the guide wire and for pulling it to remove it from the patient. In order to warn the operator that insertion of the guide wire 10 is complete and also to provide an emergency remedy in the event the entire guide wire becomes buried in the vein of a patient, the tether 12 wire extends from the end 18. Tether wire 18 is a very fine flexible stainless steel wire approximately 10 to 15 cm. in length and is an extension of the helical winding of the guide wire 10. The tether 12 is easily grasped with a clamp or, if desired, the operator may tie a loop in the end of the tether for easy withdrawal of the guide wire.

1. An angiographic guide wire having a blunt end and an operator's end, said guide wire formed of a helical winding of fine stainless wire on a fine straight wire core, said guide wire including a straight extension of said helical winding from said operator's end, said extension having a length of 10 to 15 cm. and providing a tether for said guide wire.

2. An angiographic guide wire with an included tether wire coupled to one end of said wire.
